(19) United States
(12) Patent Application Publication
(10) Pub. No.: US 2024/0140888 A1
(43) Pub. Date: May 2, 2024

(54) COMPOSITION COMPRISING AN ALKANE AND AN OXOESTER

(71) Applicant: BIOSYNTHIS, Saint Cyr Sous Dourdan (FR)

(72) Inventor: Thierry BERNOUD, Saint Cyr Sous Dourdan (FR)

(73) Assignee: BIOSYNTHIS, Saint Cyr Sous Dourdan (FR)

(21) Appl. No.: 18/231,927

(22) Filed: Aug. 9, 2023

(30) Foreign Application Priority Data
Aug. 13, 2022 (FR) 2208305

(51) Int. Cl. C07C 9/15; C07C 69/34; C11D 3/18; C11D 3/20; C11D 3/382; C11D 17/00

(52) U.S. Cl. CPC C07C 9/15; C07C 69/34; C11D 3/181; C11D 3/2093; C11D 3/382; C11D 17/0043; C11D 2111/16

(57) ABSTRACT

A composition including at least one alkane of the formula CₙH₂ₙ₊₂ with n≤12, and at least one oxoester, and to an aerosol including the same. Also, a method for degreasing, cleaning, protecting, releasing, shining, and/or lubricating a surface of an object including the application of the composition.

The present application relates to the technical field of degreasing, penetrating, cleaning, and/or lubricating compositions.

The invention relates to a composition comprising at least one alkane of the formula CₙH₂ₙ₊₂, where n≤12, and at least one oxoester, and to an aerosol comprising the same.

The invention also relates to a method for degreasing, cleaning, protecting, releasing, shining, and/or lubricating a surface comprising the application of the composition.

The product WD-40® is a multipurpose degreaser that was developed in 1953 and is widely available on the market.

The main ingredients of WD-40 are:
50% aliphatic hydrocarbons,
less than 25% petroleum-based oil,
aliphatic hydrocarbon with low vapor pressure of 12 to 18%,
2 to 3% carbon dioxide,
less than 10% inert ingredients.

Although effective, this product is harmful to the environment because its composition is of petrochemical origin. It is also highly toxic and irritating to the skin.

There is therefore a real need to have compositions for degreasing, cleaning, protecting, releasing, shining, and/or lubricating surfaces and having a better impact both ecologically and on the health of the user.

Patent application CN103510099 discloses an environmentally friendly degreasing agent. Said degreaser consists mainly of sodium carbonate and soda and particularly contains emulsifiers and silicones as minority constituents.

Other applications such as applications CN106011877, CN103614732, and CN105220166 describe work whose purpose it was to provide an environmentally friendly degreaser. None of these applications discloses the use of a composition comprising at least one alkane of the formula CₙH₂ₙ₊₂, where n≤12, and at least one oxoester.

Patent EP2726649 (or application US201412998) discloses a method for degreasing metal surfaces comprising the application of a composition comprising at least one levulinic acid ester. However, it is demonstrated in comparative tests that this composition has poor volatility, making its aerosol formulation more complex and also requiring the user to rinse or dry the surface after application. It has also been demonstrated that this composition is ineffective in treating, by soaking, the burnt greases that are commonly found in particular on mechanical parts.

Application US2007207939A1 relates to the use of fatty acid mono- and diesters of 1,3-propanediol as a solvent. It does not disclose a composition comprising at least one alkane of the formula CₙH₂ₙ₊₂, where 8≤n≤12, and at least one oxoester for use in degreasing, cleaning, protecting, releasing, shining, and/or lubricating a surface of an object.

Quite surprisingly, a method comprising the application of a composition for degreasing, cleaning, protecting, releasing, shining, and/or lubricating a surface, having good volatility, being neither toxic nor irritating to the skin, and having a good biodegradability index and degree of naturalness has been developed by the applicant.

A first object according to the present invention relates to a composition which is characterized in that it comprises:
a) at least one alkane of the formula CₙH₂ₙ₊₂, where n≤12, in a quantity of from 40% to 95% (v/v) by volume relative to the total volume of the composition, and
b) at least one oxoester of formula I,

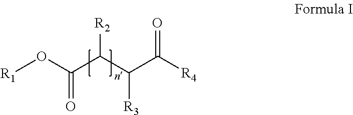

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
   the content of said oxoester being from 5% to 60% (v/v) by volume relative to the total volume of the composition.

In one embodiment, the composition comprises:
a) at least one alkane of the formula CₙH₂ₙ₊₂, where 8≤n≤12, in a quantity of from 40% to 95% (v/v) by volume relative to the total volume of the composition and
b) at least one oxoester of formula I,

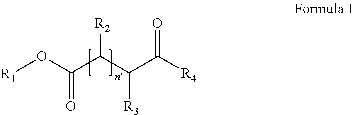

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
   the content of said oxoester being from 5% to 60% (v/v) by volume relative to the total volume of the composition.

In one embodiment, the composition according to the invention does not comprise a 1,3-propanediol fatty acid ester.

In the present application, an “alkane” is a saturated hydrocarbon consisting solely of carbon and hydrogen atoms linked together by single covalent bonds and having the general formula of CₙH₂ₙ₊₂. It is called a “linear alkane” when each carbon atom is bonded to a maximum of two carbon atoms and a “branched alkane” when certain carbon atoms are bonded to three or even four carbon atoms.

In the present application, a “bioalkane” is a biobased alkane.

For the purposes of the present application, a compound or an organic composition is understood to be “biobased” if the organic carbon that is present in the compound or composition is of 100% vegetable origin (¹⁴C) based on a radiocarbon analysis according to one of the following standards: ASTM D6866, EN 16640, or EN 16785-1.

In one embodiment, the composition according to the invention comprises a mixture of at least two alkanes of the formula CₙH₂ₙ₊₂ with different values for n, where n≤12.

In one embodiment, the composition according to the invention comprises a mixture of at least two alkanes of the formula CₙH₂ₙ₊₂ with different values for n, where 8≤n≤12

In one embodiment, at least one alkane of the formula CₙH₂ₙ₊₂, where n≤12, is a bioalkane.

In one embodiment, at least one alkane of the formula CₙH₂ₙ₊₂, where 8≤n≤12, is a bioalkane.

In one embodiment, all of the alkanes of the formula CₙH₂ₙ₊₂, where n≤12, are bioalkanes.

In one embodiment, all of the alkanes of the formula CₙH₂ₙ₊₂, where 8≤n≤12, are bioalkanes.

In one embodiment, at least one alkane of the formula CₙH₂ₙ₊₂, where n≤12, has an even number of carbon atoms.

In one embodiment, at least one alkane of the formula CₙH₂ₙ₊₂, where 8≤n≤12, has an even number of carbon atoms.

In one embodiment, all of the alkanes of the formula CₙH₂ₙ₊₂, where n≤12, have an even number of carbon atoms.

In one embodiment, all of the alkanes of the formula CₙH₂ₙ₊₂, where 8≤n≤12, have an even number of carbon atoms.

In one embodiment, at least one alkane of the formula CₙH₂ₙ₊₂, where n≤12, has an odd number of carbon atoms.

In one embodiment, at least one alkane of the formula CₙH₂ₙ₊₂, where 8≤n≤12, has an odd number of carbon atoms.

In one embodiment, at least one alkane of the formula CₙH₂ₙ₊₂, where n≤12, is selected from the group comprising: dodecane, 2-methylundecane (CAS 7045-71-8), 3-methylundecane (CAS 1002-43-3), 4-methylundecane (CAS 2980-69-0), 5-methylundecane (CAS 1632-70-8), 6-methylundecane (CAS 17302-33-9), 2.4-dimethyldecane (CAS 2801-84-5) 4,4-dimethyldecane (CAS 17312-39-9), 3,5-dimethyldecane (17312-48-0), 2,5-dimethyldecane (CAS 17312- 50-4), 2,3-c (17312-44-6), 3,3-dimethyldecane (17302-38-4), 3,7-dimethyldecane (CAS 17312-54-8), 3,4,6-trimethylnonane (CAS 62184-24-1) 3,5,6-trimethylnonane (CAS 62184-26-3), 3,5,7-trimethylnonane (CAS 62184-27-4), 2,5,7-trimethylnonane (CAS 62184-14-9), 2,5,6-trimethylnonane (CAS 62184-13-8), 2,5,7-trimethylnonane (CAS 62184-14-9), 2,5,8-trimethylnonane (CAS 49557-09-7), 3,3,4,5-tetramethyloctane (CAS 62185-21-1), 2,3,4,5-tetramethyloctane (CAS 62199-27 -3), 2,2,4,5-tetramethyloctane (CAS 62183-80-6), 2,2,5,7-tetram ethyloctane (CAS 62199-19-3), 2,3,4,7- tetramethyloctane (CAS 62199-29-5), 2,4,4,7-tetramethyloctane (CAS 35866-96-7), 3-ethyl-4-methylnonane (CAS 62184-45-6), 3-ethyl -4,5-dimethyloctane (CAS 62183-72-6), 2,5-dimethyl-6-ethyloctane (CAS 62183-50-0), undecane (CAS 1120-21-4), 2-methyldecane (CAS 6975-98-0), 4-methyldecane (CAS 2847-72-5), 3-methyldecane (13141-34-3), 5-methyldecane (CAS 13151-35-4), 2-6-dimethylnonane (CAS 17302-28-2), 3,7-dimethylnonane (CAS 17302-32-8), 4,5-dimethylnonane (CAS 17302-23-7), 2,3-dimethylnonane (CAS 2884-06-2), 2,4,6-trimethyloctane (CAS 62016-37-9), 2,5,6-trimethyloctane (CAS 62016-14-2), decane (CAS 124-18-5), 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3-dimethyloctane (CAS 7146-60-3), 2,5-dimethyloctane (CAS 15869-89-3), 3,5-dimethyloctane (CAS 15869-93-9), 4-propyl heptane (CAS 3178-29-8), 3-ethyl-2-methylheptane (CAS 14676-29-0), 2,2,3-trimethylheptane (CAS 52896-92-1), 2,3,5 trimethylheptane (CAS 20278-85-7), 2,3,6-trimethylheptane (CAS 4032- 93-3), 3,3,4-trimethylheptane (CAS 20278-87-9), 2,3,4 trimethylheptane (CAS 52896-95-4), 2,2,4 trimethylheptane (CAS 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), nonane (111-84-2), 2-methyloctane (3221-61-2), 3-methyloctane (CAS 2216-33-3), 4-methyloctane (CAS 2216-34-4), 2,4-dimethylheptane (CAS 2213-23-2), 2,6-dimethylheptane (CAS 1072-05-5), 2,3-dimethylheptane (CAS 3074 -71-3), 2,5-dimethylheptane (CAS 2216-30-0), 2,2-dimethylheptane (CAS 1071-26-7), 2,2,5-trimethylhexane (CAS 3522-94- 9), 2,3,5-trimethylhexane (CAS 1069-53-0), 2,2,4-trimethylhexane (CAS 167476-26-5), 2,3,4 trimethylhexane (CAS 921-47- 1), 3-ethylheptane (CAS 15869-80-4), 4-ethylheptane (CAS 2216-32-2), octane (CAS 111-65-9), 2,2,4-trimethylheptane (CAS 592 -27-8), 3-methylheptane (CAS 589-81-1), 2-methylheptane (CAS 592-27-8), 2,3,4-trimethylpentane (CAS 565-75-3), 2,4-dimethylhexane (CAS 589-43-5), 2,5-dimethylhexane (CAS 592-13-2), 3,4-dimethylhexane (CAS 583-48-2), 3-ethylhexane (CAS 619-99 -8), 4-methylheptane (CAS 589-53-7), and mixtures thereof.

In one embodiment, at least one alkane of the formula CₙH₂ₙ₊₂, where n≤12, is linear.

In one embodiment, all of the alkanes of the formula CₙH₂ₙ₊₂, where n≤12, are linear.

In one embodiment, at least one alkane of the formula CₙH₂ₙ₊₂, where n≤12, is branched.

In one embodiment, all of the alkanes of the formula CₙH₂ₙ₊₂, where n≤12, are branched.

In one embodiment, the at least one alkane of the formula CₙH₂ₙ₊₂, where n≤12, is selected from the group consisting of dodecane, undecane, decane, nonane, and/or an octane.

In one embodiment, at least one alkane of the formula CₙH₂ₙ₊₂, where n≤12, is a dodecane.

In one embodiment, at least one alkane of the formula CₙH₂ₙ₊₂, where n≤12, is an undecane.

In one embodiment, at least one alkane of the formula CₙH₂ₙ₊₂, where n≤12, is a decane.

In one embodiment, at least one alkane of the formula CₙH₂ₙ₊₂, where n≤12, is a nonane.

In one embodiment, at least one alkane of the formula CₙH₂ₙ₊₂, where n≤12, is an octane.

In one embodiment, the alkanes of the formula CₙH₂ₙ₊₂, where n≤12, comprise a mixture of decane and dodecane.

In one embodiment, the alkanes of the formula CₙH₂ₙ₊₂, where n≤12, consist of a mixture of decane and dodecane.

In one embodiment, at least one alkane of the formula CₙH₂ₙ₊₂ where 8≤n≤12, is linear.

In one embodiment, all of the alkanes of the formula CₙH₂ₙ₊₂, where 8≤n≤12, are linear.

In one embodiment, at least one alkane of the formula CₙH₂ₙ₊₂, where 8≤n≤12, is branched.

In one embodiment, all of the alkanes of the formula CₙH₂ₙ₊₂, where 8≤n≤12, are branched.

In one embodiment, the at least one alkane of the formula CₙH₂ₙ₊₂, where 8≤n≤12, is selected from the group consisting of dodecane, undecane, decane, nonane, and/or an octane.

In one embodiment, at least one alkane of the formula CₙH₂ₙ₊₂, where 8≤n≤12, is a dodecane.

In one embodiment, at least one alkane of the formula CₙH₂ₙ₊₂, where 8≤n≤12, is an undecane.

In one embodiment, at least one alkane of the formula CₙH₂ₙ₊₂, where 8≤n ≤12, is a decane.

In one embodiment, at least one alkane of the formula CₙH₂ₙ₊₂, where 8≤n≤12, is a nonane.

In one embodiment, at least one alkane of the formula CₙH₂ₙ₊₂, where 8≤n≤12, is an octane.

In one embodiment, the alkanes of the formula CₙH₂ₙ₊₂, where 8≤n≤12, comprise a mixture of decane and dodecane.

In one embodiment, the alkanes of the formula CₙH₂ₙ₊₂, where 8≤n≤12, consist of a mixture of decane and dodecane.

The “naturalness index” of an organic composition is the percentage of organic carbon of vegetable origin as determined by radiocarbon analysis (¹⁴C) according to one of the following standards: ASTM D6866, EN 16640, or EN 16785-1.

Within the meaning of the present invention, the OECD 301F method makes it possible to assess the biodegradability of a substance using a manometric respirometry test.

Within the meaning of the present invention, the OECD 301F method makes it possible to assess the biodegradability of a substance using a manometric respirometry test.

In one embodiment, the alkane of the formula CₙH₂ₙ₊₂, where n≤12, has a naturalness index of greater than or equal to 60% as determined by the carbon 14 (¹⁴C) content according to one of the following standards: ASTM D6866, EN 16640, or EN 16785-1

In one embodiment, the alkane of the formula CₙH₂ₙ₊₂, where n≤12, has a naturalness index of greater than or equal to 70% as determined by the carbon 14 (¹⁴C) content according to one of the following standards: ASTM D6866, EN 16640, or EN 16785-1

In one embodiment, the alkane of the formula CₙH₂ₙ₊₂, where n≤12, has a naturalness index of greater than or equal to 75% as determined by the carbon 14 (¹⁴C) content according to one of the following standards: ASTM D6866, EN 16640, or EN 16785-1

In one embodiment, the alkane of the formula CₙH₂ₙ₊₂, where n≤12, has a naturalness index of greater than or equal to 85% as determined by the carbon 14 (¹⁴C) content according to one of the following standards: ASTM D6866, EN 16640, or EN 16785-1

In one embodiment, the alkane of the formula CₙH₂ₙ₊₂, where n≤12, has a naturalness index of greater than or equal to 90% as determined by the carbon 14 (¹⁴C)) content according to one of the following standards: ASTM D6866, EN 16640, or EN 16785-1

Advantageously, the alkane of the formula CₙH₂ₙ₊₂, where n≤12, has a naturalness index of 100% as determined by the carbon 14 (¹⁴C) content according to one of the following standards: ASTM D6866, EN 16640, or EN 16785-1

In one embodiment, the alkane of the formula CₙH₂ₙ₊₂, where n≤12, is obtained from raw materials of vegetable, bacterial, or animal origin.

In one embodiment, the alkane of the formula CₙH₂ₙ₊₂, where n≤12, is obtained from raw materials of vegetable origin.

In one embodiment, the alkane of the formula CₙH₂ₙ₊₂, where n≤12, has a biodegradability of at least 50% according to the OECD 301F method.

In one embodiment, the alkane of the formula CₙH₂ₙ₊₂, where n≤12, has a biodegradability of at least 60% according to the OECD 301F method.

In one embodiment, the alkane of the formula CₙH₂ₙ₊₂, where n≤12, has a biodegradability of 50 to 80% according to the OECD 301F method.

In one embodiment, the alkane of the formula CₙH₂ₙ₊₂, where n≤12, has a biodegradability of 50 to 70% according to the OECD 301F method.

In one embodiment, the alkane of the formula CₙH₂ₙ₊₂, where n≤12, has a biodegradability of 50 to 60% according to the OECD 301F method.

In one embodiment, the alkane of the formula CₙH₂ₙ₊₂, where n≤12, has a biodegradability of 60 to 70% according to the OECD 301F method.

In one embodiment, the alkane of the formula CₙH₂ₙ₊₂, where n≤12, has a biodegradability of 70 to 80% according to the OECD 301F method.

In one embodiment, the alkane of the formula CₙH₂ₙ₊₂, where 8≤n≤12, has a naturalness index of greater than or equal to 60% as determined by the carbon 14 (¹⁴C) content according to one of the following standards: ASTM D6866, EN 16640, or EN 16785-1

In one embodiment, the alkane of the formula CₙH₂ₙ₊₂, where 8≤n≤12, has a naturalness index of greater than or equal to 70% as determined by the carbon 14 (¹⁴C) content according to one of the following standards: ASTM D6866, EN 16640, or EN 16785-1

In one embodiment, the alkane of the formula CₙH₂ₙ₊₂, where 8≤n≤12, has a naturalness index of greater than or equal to 75% as determined by the carbon 14 (¹⁴C) content according to one of the following standards: ASTM D6866, EN 16640, or EN 16785-1

In one embodiment, the alkane of the formula CₙH₂ₙ₊₂, where 8≤n≤12, has a naturalness index of greater than or equal to 85% as determined by the carbon 14 (¹⁴C) content according to one of the following standards: ASTM D6866, EN 16640, or EN 16785-1

In one embodiment, the alkane of the formula CₙH₂ₙ₊₂, where 8≤n≤12, has a naturalness index of greater than or equal to 90% as determined by the carbon 14 (¹⁴C) content according to one of the following standards: ASTM D6866, EN 16640, or EN 16785-1

Advantageously, the alkane of the formula CₙH₂ₙ₊₂, where 8≤n≤12, has a naturalness index of 100% as determined by the carbon 14 (¹⁴C) content according to one of the following standards: ASTM D6866, EN 16640, or EN 16785-1

In one embodiment, the alkane of the formula CₙH₂ₙ₊₂, where 8≤n≤12, is obtained from raw materials of vegetable, bacterial, or animal origin.

In one embodiment, the alkane of the formula CₙH₂ₙ₊₂, where 8≤n≤12, is obtained from raw materials of vegetable origin.

In one embodiment, the alkane of the formula CₙH₂ₙ₊₂, where 8≤n≤12, has a biodegradability of at least 50% according to the OECD 301F method.

In one embodiment, the alkane of the formula CₙH₂ₙ₊₂, where 8≤n≤12, has a biodegradability of at least 60% according to the OECD 301F method.

In one embodiment, the alkane of the formula CₙH₂ₙ₊₂, where 8≤n≤12, has a biodegradability of 50 to 80% according to the OECD 301F method.

In one embodiment, the alkane of the formula CₙH₂ₙ₊₂, where 8≤n≤12, has a biodegradability of 50 to 70% according to the OECD 301F method.

In one embodiment, the alkane of the formula CₙH₂ₙ₊₂, where 8≤n≤12, has a biodegradability of 50 to 60% according to the OECD 301F method.

In one embodiment, the alkane of the formula CₙH₂ₙ₊₂, where 8≤n≤12, has a biodegradability of 60 to 70% according to the OECD 301F method.

In one embodiment, the alkane of the formula CₙH₂ₙ₊₂, where 8≤n≤12, has a biodegradability of 70 to 80% according to the OECD 301F method.

In one embodiment, the composition according to the invention comprises at least one oxoester of formula II:

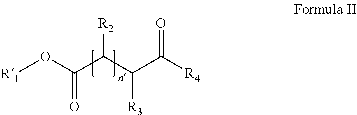

in which
R′₁ is selected from the group consisting of linear or branched alkyls comprising from 1 to 4 carbon atoms;
R₂, R₃, and R₄, which are identical or different, are selected from the group consisting of the hydrogen atom or linear or branched alkyls comprising from 1 to 4 carbon atoms; and
n′ is a natural number between 1 and 4.

In one embodiment, the composition according to the invention comprises at least one oxoester of formula II, in which R₂ and R₃, which are identical or different, are selected from the group consisting of the hydrogen atom, the methyl group or the ethyl group, R₄ is selected from the group consisting of linear or branched alkyls comprising from 1 to 3 carbon atoms, and n′ is a natural number between 1 and 3.

In one embodiment, the composition according to the invention comprises at least one oxoester of formula I, in which R₂ and R₃ are a hydrogen atom, R4 is a methyl group, and n′ is equal to 1.

In one embodiment, the composition according to the invention comprises at least one oxoester of formula II, in which R₂ and R₃ are a hydrogen atom, R₄ is a methyl group, and n′ is equal to 1.

In one embodiment, the composition according to the invention comprises an oxoester alone or as a mixture.

In one embodiment, the oxoester alone or as a mixture is selected from the group comprising: methyl levulinate (CAS 624-45-3), ethyl levulinate (CAS 539-88-8), propyl levulinate (645-67-0), isopropyl levulinate (CAS 21884-26-4), butyl levulinate (CAS 2052-15-5), isobutyl levulinate (CAS 3757-32-2), tert-butyl levulinate (CAS 2854-10-6), s-butyl levulinate (CAS 85734-01-6), penthyl levulinate (CAS 20279-49-6) hexyl levulinate (CAS 24431-34-3), octyl levulinate (CAS 41780-57-8), 2-methyl-4-oxovaleric acid ethyl ester (CAS 4749-12-6), methyl 6-oxoheptanoate (CAS 2046-21-1), methyl 4-oxohexanoate (CAS 2955-62-6), methyl 5-oxohexanoate (CAS 13984-50-4), methyl 3-methyl -5-oxohexanoate (CAS 14983-18-7), 5-ketoenanthic acid methyl ester (17745-32-3), methyl ester (CAS 36045-56-4), methyl 2-ethyl-4-oxopentanoate (CAS 62359-06-2), methyl 3-methyl-4-oxohexanoate (CAS 69448-35-7), 2-methyl-5-oxohexanoic acid methyl ester (CAS 38872-30-9), 3-methyl-4-oxopentanoic acid ethyl ester (CAS 55424-74-3), 2-methyl-4-oxohexanoic acid methyl ester (CAS 75436-59-8), methyl (2S,4R)-2,4-dimethyl-5-oxohexanoate (CAS 93176-58-0), 4-oxopentanoic acid-2-propylmethyl ester (CAS 244196-06-3), 2,5-dimethyl-4-oxohexanoic acid methyl ester (CAS 1249353-11-4), 4-oxo-octanoic acid methyl ester (CAS: 4316-48-7), 2-methyl-6-oxoheptanoic acid methyl ester (CAS 2570-90-3), 3-methyl-6-oxoheptanoic acid methyl ester (CAS 5128-55-2), 6-methyl-5-oxoheptanoic acid methyl ester (CAS 23575-33-9), 3-methyl-5-oxohexanoic acid ethyl ester (CAS 38052-21-0), 2-methyl-5-oxoheptanoic acid methyl ester (CAS 25912-38-3), 4-methyl-6-oxoheptanoic acid methyl ester (CAS 41841-53-6), 5-methyl-4-oxoheptanoic acid methyl ester (CAS 42511-74-0), 4-methyl-5-oxoheptanoic acid methyl ester (CAS 54225-40-0), 3,5-dimethyl-4-oxohexanoic acid methyl ester (CAS 64712-02-3), 6-methyl-4-oxohexanoic acid methyl ester (CAS 64712-02-3), heptanoic acid (CAS 76678-33-6), 2-methyl-4-oxoheptanoic acid methyl ester (CAS 90647-21-5), 4-ethyl-5-oxohexanoic acid methyl ester (CAS 90647-24-8), 3-methyl-5-oxoheptanoic acid methyl ester (CAS 103252-99-9), 2-ethyl-5-oxohexanoic acid methyl ester (CAS 103260-39-5), hexanoic acid 3-acetyl methyl ester (CAS 1081559-93-4), 5-methyl-6-oxoheptanoic acid methyl ester (CAS 344295-02-9), 3-methyl-4-oxoheptanoic acid methyl ester (CAS 64712-01-2), methyl 2-(1-methylethyl)-4-oxopentaoate (CAS 99183-33-2), methyl 2,3-dimethyl-4-oxohexanoate (CAS 86044-19-1), ethyl 2-ethyl-4-oxopentanoate (CAS 101514-30-1), 5-oxohexanoic acid ethyl ester (CAS 13984 -57-1), ethyl 3-methyl-4-oxohexanoate (CAS 42895-72-7), ethyl 2,3-dimethyl-4-oxopentanoate (CAS 136964-44-8), 4-oxohexanoic acid ethyl ester (CAS 3249-33-0), 6-oxoheptanoic acid ethyl ester (CAS 30956-41-3), 1-methylethyl 4-oxohexanoate (CAS 939422-07-8), 2-ethyl-4-oxopentanoic acid ethyl ester (CAS 101514-30-1), 2-ethyl-5-methyl-4-oxohexanoic acid methyl ester (CAS 1195311-69-3).

In one embodiment, at least one oxoester is a levulinate.

In one embodiment, at least one oxoester is a levulinate selected from the group comprising: methyl levulinate (CAS 624-45-3), ethyl levulinate (CAS 539-88-8), propyl levulinate (645-67-0), isopropyl levulinate (CAS 21884-26-4), butyl levulinate (CAS 2052-15-5), isobutyl levulinate (CAS 3757-32-2), tert-butyl levulinate (CAS 2854-10-6), s-butyl levulinate (CAS 85734-01-6), penthyl levulinate (CAS 20279-49-6) hexyl levulinate (CAS 24431-34-3), octyl levulinate (CAS 41780-57-8), or mixtures thereof.

In one embodiment, at least one oxoester is methyl levulinate.

In one embodiment, at least one oxoester is butyl levulinate.

In one embodiment, at least one oxoester is ethyl levulinate.

In one embodiment, at least one oxoester is butyl levulinate and/or ethyl levulinate.

In one embodiment, the oxoester comprises ethyl levulinate and/or butyl levulinate.

In one embodiment, the oxoester consists of an ethyl levulinate and/or a butyl levulinate.

In one embodiment, the oxoester has a molecular mass of less than or equal to 200 g/mol.

In one embodiment, the oxoester has a molecular mass of less than or equal to 180 g/mol.

In one embodiment, the oxoester has a molecular mass of less than or equal to 160 g/mol.

In one embodiment, the oxoester has a molecular mass of less than or equal to 150 g/mol.

In one embodiment, the composition according to the invention comprises a decane and an ethyl levulinate.

In one embodiment, the composition according to the invention comprises a dodecane and an ethyl levulinate.

In one embodiment, the composition according to the invention comprises a decane, a dodecane, and an ethyl levulinate.

In one embodiment, the composition according to the invention comprises a branched alkane with 10 carbon atoms and an ethyl levulinate.

In one embodiment, the composition according to the invention comprises a branched alkane with 12 carbon atoms and an ethyl levulinate.

In one embodiment, the composition according to the invention comprises a decane and a butyl levulinate.

In one embodiment, the composition according to the invention comprises a dodecane and a butyl levulinate.

In one embodiment, the composition according to the invention comprises a decane, a dodecane, and a butyl levulinate.

In one embodiment, the composition according to the invention comprises a branched alkane with 10 carbon atoms and a butyl levulinate.

In one embodiment, the composition according to the invention comprises a branched alkane with 12 carbon atoms and a butyl levulinate.

In one embodiment, the composition according to the invention comprises a decane, a dodecane, an ethyl levulinate, and a butyl levulinate.

In one embodiment, the oxoester has a naturalness index of greater than or equal to 60% as determined by the carbon 14 (¹⁴C) content according to one of the following standards: ASTM D6866, EN 16640, or EN 16785-1

In one embodiment, the oxoester has a naturalness index of greater than or equal to 70% as determined by the carbon 14 (14C) content according to one of the following standards: ASTM D6866, EN 16640, or EN 16785-1

In one embodiment, the oxoester has a naturalness index of greater than or equal to 75% as determined by the carbon 14 (14C) content according to one of the following standards: ASTM D6866, EN 16640, or EN 16785-1

In one embodiment, the oxoester has a naturalness index of greater than or equal to 85% as determined by the carbon 14 (14C) content according to one of the following standards: ASTM D6866, EN 16640, or EN 16785-1

In one embodiment, the oxoester has a naturalness index of greater than or equal to 90% as determined by the carbon 14 (14C) content according to one of the following standards: ASTM D6866, EN 16640, or EN 16785-1

Advantageously, the oxoester has a naturalness index of 100% as determined by the carbon 14 (¹⁴C) content according to one of the following standards: ASTM D6866, EN 16640, or EN 16785-1

In one embodiment, the oxoester is obtained from raw materials of vegetable, bacterial, or animal origin.

In one embodiment, the levulinate is obtained is derived from biomass.

In one embodiment, the oxoester has a biodegradability of at least 50% according to the OECD 301F method.

In one embodiment, the oxoester has a biodegradability of at least 60% according to the OECD 301F method.

In one embodiment, the oxoester has a biodegradability of 50% to 80% according to the OECD 301F method.

In one embodiment, the oxoester has a biodegradability of 50% to 70% according to the OECD 301F method.

In one embodiment, the oxoester has a biodegradability of 50% to 60% according to the OECD 301F method.

In one embodiment, the oxoester has a biodegradability of 60 to 70% according to the OECD 301F method.

In one embodiment, the oxoester has a biodegradability of 70 to 80% according to the OECD 301F method.

In one embodiment, the composition according to the invention comprises a content of an oxoester of formula I of from 10% to 50% (v/v) by volume relative to the total volume of the composition according to the invention.

In one embodiment, the composition according to the invention comprises a content of an oxoester of formula I of from 10% to 30% (v/v) relative to the total volume of the composition according to the invention.

In one embodiment, the composition according to the invention comprises a content of an oxoester of formula I of from 10% to 20% (v/v) relative to the total volume of the composition according to the invention.

In one embodiment, the composition according to the invention comprises a butyl levulinate content of at least 1% (v/v) by volume relative to the total volume of the composition according to the invention.

In one embodiment, the composition according to the invention comprises a butyl levulinate content of at least 5% (v/v) by volume relative to the total volume of the composition according to the invention.

In one embodiment, the composition according to the invention comprises a butyl levulinate content of from 1 to 60% (v/v) by volume relative to the total volume of the composition according to the invention.

In one embodiment, the composition according to the invention comprises a butyl levulinate content of from 5 to 25% (v/v) by volume relative to the total volume of the composition according to the invention.

In one embodiment, the composition according to the invention comprises a butyl levulinate content of approximately 5% (v/v) by volume relative to the total volume of the composition according to the invention.

In one embodiment, the composition according to the invention comprises an ethyl levulinate content of at least 1% (v/v) by volume relative to the total volume of the composition according to the invention.

In one embodiment, the composition according to the invention comprises an ethyl levulinate content of at least 5% (v/v) by volume relative to the total volume of the composition according to the invention.

In one embodiment, the composition according to the invention comprises an ethyl levulinate content of from 1% to 60% (v/v) by volume relative to the total volume of the composition according to the invention.

In one embodiment, the composition according to the invention comprises an ethyl levulinate content of from 5% to 25% (v/v) by volume relative to the total volume of the composition according to the invention.

In one embodiment, the composition according to the invention comprises an ethyl levulinate content of approximately 5% (v/v) by volume relative to the total volume of the composition according to the invention.

In one embodiment, the composition according to the invention comprises an alkane content of the formula CₙH₂ₙ₊₂, where n≤12, of from 50% to 90% (v/v) by volume relative to the total volume of the composition according to the invention.

In one embodiment, the composition according to the invention comprises an alkane content of the formula CₙH₂ₙ₊₂, where n≤12, of from 70% to 90% (v/v) relative to the total volume of the composition according to the invention.

In one embodiment, the composition according to the invention comprises an alkane content of the formula CₙH₂ₙ₊₂, where n≤12, of from 80% to 90% (v/v) relative to the total volume of the composition according to the invention.

In one embodiment, the composition according to the invention comprises an alkane content of the formula CₙH₂ₙ₊₂, where 8≤n≤12, of from 50% to 90% (v/v) by volume relative to the total volume of the composition according to the invention.

In one embodiment, the composition according to the invention comprises an alkane content of the formula CₙH₂ₙ₊₂, where 8≤n≤12, of from 70% to 90% (v/v) relative to the total volume of the composition according to the invention.

In one embodiment, the composition according to the invention comprises an alkane content of the formula CₙH₂ₙ₊₂, where 8≤n≤12, of from 80% to 90% (v/v) relative to the total volume of the composition according to the invention.

In one embodiment, the composition according to the invention comprises a decane content of at least 1% (v/v) by volume relative to the total volume of the composition according to the invention.

In one embodiment, the composition according to the invention comprises a decane content of at least 5% (v/v) by volume relative to the total volume of the composition according to the invention.

In one embodiment, the composition according to the invention comprises a decane content of from 1% to 50% (v/v) by volume relative to the total volume of the composition according to the invention.

In one embodiment, the composition according to the invention comprises a decane content of from 5% to 30% (v/v) by volume relative to the total volume of the composition according to the invention.

In one embodiment, the composition according to the invention comprises a decane content of from 7.5% to 20% (v/v) by volume relative to the total volume of the composition according to the invention.

In one embodiment, the composition according to the invention comprises a dodecane content of at least 20% (v/v) by volume relative to the total volume of the composition according to the invention.

In one embodiment, the composition according to the invention comprises a dodecane content of at least 30% (v/v) by volume relative to the total volume of the composition according to the invention.

In one embodiment, the composition according to the invention comprises a dodecane content of from 30% to 80% (v/v) by volume relative to the total volume of the composition according to the invention.

In one embodiment, the composition according to the invention comprises a dodecane content of from 40% to 75% (v/v) by volume relative to the total volume of the composition according to the invention.

In one embodiment, the composition according to the invention comprises a proportion of decane of from 5% to 30% (v/v) by volume relative to the total volume of the alkanes of the formula CₙH₂ₙ₊₂, where n≤12.

In one embodiment, the composition according to the invention comprises a proportion of decane of from 10% to 20% (v/v) by volume relative to the total volume of the alkanes of the formula CₙH₂ₙ₊₂, where n≤12.

In one embodiment, the composition according to the invention comprises a proportion of dodecane of from 70% to 95% (v/v) by volume relative to the total volume of the alkanes of the formula CₙH₂ₙ₊₂, where n≤12.

In one embodiment, the composition according to the invention comprises a proportion of dodecane of from 80% to 90% (v/v) by volume relative to the total volume of the alkanes of the formula CₙH₂ₙ₊₂, where n≤12.

In one embodiment, the composition according to the invention comprises a proportion of decane of from 5% to 30% (v/v) by volume relative to the total volume of the alkanes of the formula CₙH₂ₙ₊₂, where 8≤n≤12.

In one embodiment, the composition according to the invention comprises a proportion of decane of from 10% to 20% (v/v) by volume relative to the total volume of the alkanes of the formula CₙH₂ₙ₊₂, where 8≤n≤12.

In one embodiment, the composition according to the invention comprises a proportion of dodecane of from 70% to 95% (v/v) by volume relative to the total volume of the alkanes of the formula CₙH₂ₙ₊₂, where 8≤n≤12.

In one embodiment, the composition according to the invention comprises a proportion of dodecane of from 80% to 90% (v/v) by volume relative to the total volume of the alkanes of the formula CₙH₂ₙ₊₂, where 8≤n≤12.

In one embodiment, the composition according to the invention does not comprise petrochemical derivatives.

In one embodiment, the composition does not comprise shea and/or shea extract.

In one embodiment, the composition according to the invention also comprises at least one oil.

Another object according to the present invention thus relates to a composition which is characterized in that it comprises:
a) at least one alkane of the formula CₙH₂ₙ₊₂, where n≤12, in a quantity of from 40% to 95% (v/v) by volume relative to the total volume of the composition and
at least one oxoester of formula I:

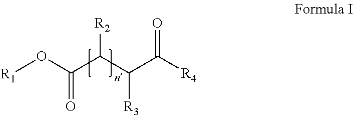

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
in a quantity of from 5% to 60% (v/v) by volume relative to the total volume of the composition according to the invention.
b) at least one oil.

Another object according to the present invention thus relates to a composition which is characterized in that it comprises:
a) at least one alkane of the formula CₙH₂ₙ₊₂, where 8≤n≤12, in a quantity of from 40% to 95% (v/v) by volume relative to the total volume of the composition and
b) at least one oxoester of formula I:

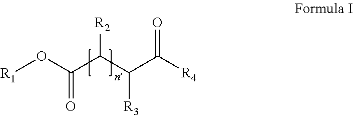

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
in a quantity of from 5% to 60% (v/v) by volume relative to the total volume of the composition according to the invention.
c) at least one oil.
   In one embodiment of the composition comprising at least one oil, the at least one oxoester is selected from the oxoesters of formula II:

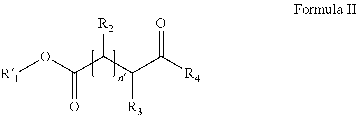

in which
R′₁ is selected from the group consisting of linear or branched alkyls comprising from 1 to 4 carbon atoms;
R₂, R₃, and R₄, which are identical or different, are selected from the group consisting of the hydrogen atom or linear or branched alkyls comprising from 1 to 4 carbon atoms; and
n′ is a natural number between 1 and 4.

In one embodiment of the composition comprising at least one oil, the at least one oxoester is selected from the oxoesters of formula I, in which R₂ and R₃ are a hydrogen atom, R₄ is a methyl group, and n′ is equal to 1.

In one embodiment of the composition comprising at least one oil, the at

least one oxoester is selected from the oxoesters of formula II, in which R₂ and R₃, which are identical or different, are selected from the group consisting of the hydrogen atom, the methyl group or the ethyl group, R₄ is selected from the group consisting of linear or branched alkyls comprising from 1 to 3 carbon atoms, and n′ is a natural number between 1 and 3.

In one embodiment of the composition comprising at least one oil, the at least one oxoester is selected from the oxoesters of formula II, in which R₂ and R₃ are a hydrogen atom, R4 is a group methyl, and n′ is equal to 1.

The presence of an oil in the composition has the advantage of imparting lubricating properties to the composition. It also has the advantage of depositing a protective film on the surface, preventing fouling. Finally, the presence of oil makes it possible to increase the degreasing power of the composition.

In one embodiment, the oil comprises an oil of vegetable and/or synthetic origin.

In one embodiment, the composition according to the invention does not comprise mineral oil.

In one embodiment, the oil comprises a hydrocarbon, silicone, fluorinated, and/or fluorosilicone oil.

Within the meaning of the present invention, hydrocarbon oil is understood to be an oil consisting substantially of carbon, hydrogen, and optionally oxygen and/or nitrogen atoms.

In one embodiment, these hydrocarbon oils are selected from the group of linear or branched alkanes of mineral or synthetic origin, such as linear or branched alkanes with 17 to 40 carbon atoms, (poly)esters and (poly)ethers, and in particular (poly)esters of C₂-C₂₄ (preferably C₆-C₂₀) acids, C₆-C₂₀ fatty acid triglycerides, vegetable oils, dialkyl carbonates, branched and/or unsaturated fatty acids and/or branched and/or unsaturated fatty alcohols and mixtures thereof.

In one embodiment, the silicone oils are selected from the group comprising phenyl silicone oils, non-volatile polydimethylsiloxanes, or derivatives of non-volatile polydimethylsiloxanes and mixtures thereof.

In one embodiment, the oil comprises an oil selected from the group consisting of non-volatile linear or branched alkanes.

In one embodiment, the non-volatile linear or branched alkanes are selected from the group consisting of linear or branched alkanes with 17 to 40 carbon atoms.

In one embodiment, the oil comprises an oil of vegetable origin.

In one embodiment, the oil comprises a vegetable hydrocarbon oil.

In one embodiment, the oil comprises a vegetable hydrocarbon oil selected from the group comprising wheat germ, sunflower, grapeseed, sesame, coconut, apricot, corn, avocado, soy, olive, sweet almond, cottonseed, palm, macadamia, rapeseed, jojoba, hazelnut, poppy, alfalfa, pumpkin, blackcurrant, squash, evening primrose, and barley oils and mixtures thereof.

In one embodiment, the composition according to the invention does not contain castor oil.

In one embodiment, the oil comprises a coconut oil.

In one embodiment, the oil is coconut oil.

In one embodiment, the composition according to the invention has an oil content of less than or equal to 30% (v/v) by volume of oil relative to the total volume of the composition according to the invention.

In one embodiment, the composition according to the invention has an oil content of less than or equal to 20% (v/v) by volume of oil relative to the total volume of the composition according to the invention.

In one embodiment, the composition according to the invention has an oil content of less than or equal to 15% (v/v) by volume of oil relative to the total volume of the composition according to the invention.

In one embodiment, the composition according to the invention has a lower oil content of from 5 to 20% (v/v) by volume of oil relative to the total volume of the composition according to the invention.

In one embodiment, the composition according to the invention has a naturalness index of greater than or equal to 60% as determined by the carbon 14 (¹⁴C) content according to one of the following standards: ASTM D6866, EN 16640, or EN 16785-1

In one embodiment, the composition according to the invention has a naturalness index of greater than or equal to 70% as determined by the carbon 14 (¹⁴C)) content according to one of the following standards: ASTM D6866, EN 16640, or EN 16785-1

In one embodiment, the composition according to the invention has a naturalness index of greater than or equal to 75% as determined by the carbon 14 (¹⁴C) content according to one of the following standards: ASTM D6866, EN 16640, or EN 16785-1

In one embodiment, the composition according to the invention has a naturalness index of greater than or equal to 85% as determined by the carbon 14 (¹⁴C)) content according to one of the following standards: ASTM D6866, EN 16640, or EN 16785-1

In one embodiment, the composition according to the invention has a naturalness index of greater than or equal to 90% as determined by the carbon 14 (¹⁴C) content according to one of the following standards: ASTM D6866, EN 16640, or EN 16785-1

Advantageously, the composition according to the invention has a naturalness index of 100% as determined by the carbon 14 (¹⁴C) content according to one of the following standards: ASTM D6866, EN 16640, or EN 16785-1

In one embodiment, the composition according to the invention is obtained from raw materials of vegetable, bacterial, or animal origin.

In one embodiment, the composition according to the invention has a biodegradability of at least 50% according to the OECD 301F method.

In one embodiment, the composition according to the invention has a biodegradability of at least 60% according to the OECD 301F method.

In one embodiment, the composition according to the invention has a biodegradability of from 50% to 80% according to the OECD 301F method.

In one embodiment, the composition according to the invention has a biodegradability of from 50% to 70% according to the OECD 301F method.

In one embodiment, the composition according to the invention has a biodegradability of from 50% to 60% according to the OECD 301F method.

In one embodiment, the composition according to the invention has a biodegradability of from 60% to 70% according to the OECD 301F method.

In one embodiment, the composition according to the invention has a biodegradability of from 70% to 80% according to the OECD 301F method.

In one embodiment, the composition according to the invention has a density of from 0.6 to 1 g/cm³.

In one embodiment, the composition according to the invention has a density of from 0.75 to 0.85 g/cm³.

In one embodiment, the composition according to the invention has a dynamic viscosity of from 1 to 3 centipoise.

In one embodiment, the composition according to the invention has a dynamic viscosity of from 1.5 to 2.75 centipoise.

In one embodiment, the composition according to the invention has a kinematic viscosity of from 1.5 to 3 centistokes.

In one embodiment, the composition according to the invention has a kinematic viscosity of from 1.8 to 2.6 centistokes.

The invention also relates to an aerosol comprising the composition according to the invention and a propellant gas.

In one embodiment, the propellant gas is selected from the group of carbon dioxide (CO₂), butane, propane, isobutane, dimethyl ether, nitrogen, nitrous oxide, or mixtures thereof.

In one embodiment, the propellant gas is selected from the group of carbon dioxide (CO₂), butane, propane, or mixtures thereof.

In one embodiment, the propellant gas is carbon dioxide.

In one embodiment, the aerosol according to the invention is packaged in an aerosol can.

In one embodiment, the internal pressure of the aerosol can is at least 7 bar.

In one embodiment, the internal pressure of the aerosol can is at least 8 bar.

In one embodiment, the aerosol can comprises a nozzle that enables wide mist diffusion of at least 20 cm.

In one embodiment, the aerosol can comprises a nozzle that enables wide mist diffusion of at least 30 cm.

In one embodiment, the aerosol can allows a flow rate of at least 1 gram per second.

In one embodiment, the aerosol can allows a flow rate of at least 2 grams per second.

In one embodiment, the aerosol can allows a flow rate of at least 4 grams per second.

Another object according to the present invention relates to a method for degreasing, cleaning, protecting, releasing, shining, and/or lubricating a surface of an object comprising at least one step of applying to said surface a composition comprising:
a) At least one alkane of the formula CₙH₂ₙ₊₂, where n≤12, and at least one oxoester of formula I,

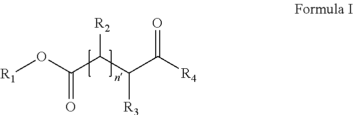

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4.

In one embodiment, the method for degreasing, cleaning, protecting, releasing, shining, and/or lubricating a surface of an object according to the invention comprises at least one step of applying to said surface a composition comprising:
a) at least one alkane of the formula CₙH₂ₙ₊₂, where 8≤n≤12, and
b) at least one oxoester of formula I,

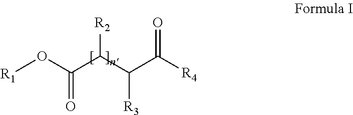

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4.

In one embodiment, the method for degreasing, cleaning, protecting, releasing, shining, and/or lubricating a surface of an object according to the invention comprises at least one step of applying to said surface a composition comprising:
a) at least one alkane of the formula CₙH₂ₙ₊₂, where 8≤n≤12, and
b) at least one oxoester of formula I,

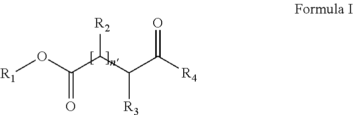

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
   and in which the alkane of the formula CₙH₂ₙ₊₂, where 8≤n≤12, is linear or branched and selected from the group consisting of dodecane, undecane, decane, nonane, and/or an octane.

In one embodiment, the method for degreasing, cleaning, protecting, releasing, shining, and/or lubricating a surface of an object according to the invention comprises at least one step of applying to said surface a composition comprising:
a) at least one alkane of the formula CₙH₂ₙ₊₂, where 8≤n≤12, and
b) at least one oxoester of formula I,

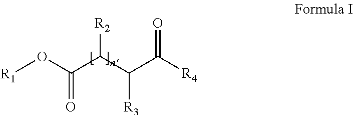

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
   and in which the alkane of the formula CₙH₂ₙ₊₂, where 8≤n≤12, is linear or branched and selected from the group consisting of dodecane, undecane, decane, nonane, and/or an octane.

In one embodiment, the method for degreasing, cleaning, protecting, releasing, shining, and/or lubricating a surface of an object according to the invention comprises at least one step of applying to said surface a composition comprising:
a) at least one alkane of the formula CₙH₂ₙ₊₂, where 8≤n≤12, and
b) at least one oxoester of formula I,

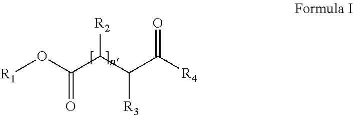

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
   and in which the alkane of the formula CₙH₂ₙ₊₂, where 8≤n≤12, is selected from the group comprising: dodecane, 2-methylundecane (CAS 7045-71-8), 3-methylundecane (CAS 1002 -43-3), 4-methylundecane (CAS 2980-69-0), 5-methylundecane (CAS 1632-70-8), 6-methylundecane (CAS 17302-33-9), 2,4-dimethyldecane (CAS 2801-84-5) 4,4-dimethyldecane (CAS 17312-39-9), 3,5-dimethyldecane (17312-48-0), 2,5-dimethyldecane (CAS 17312-50- 4), 2,3-dimethyldecane (17312-44-6), 3,3-dimethyldecane (17302-38-4), 3,7-dimethyldecane (CAS 17312-54-8), 3,4,6-trimethylnonane (CAS 62184-24-1) 3,5,6-trimethylnonane (CAS 62184-26-3), 3,5,7-trimethylnonane (CAS 62184-27-4), 2.5, 7-trimethylnonane (CAS 62184-14-9), 2,5,6-trimethylnonane (CAS 62184-13-8), 2,5,7-trimethylnonane (CAS 62184-14-9), 2.5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7), 3-ethyl-4-methylnonane (CAS 62184-45-6), 3-ethyl-4,5-dimethyloctane (CAS 62183-72-6), 2,5-dimethyl-6-ethyloctane (CAS 62183-50-0), undecane (CAS 1120-21-4), 2-methyldecane (CAS 6975 -98-0), 4-methyldecane (CAS 2847-72-5), 3-methyldecane (13141-34-3), 5-methyldecane (CAS 13151-35-4), 2-6-dimethylnonane (CAS 17302-28-2), 3,7-dimethylnonane (CAS 17302-32-8), 4,5-dimethylnonane (CAS 17302-23-7), 2,3-dimethylnonane (CAS 2884- 06-2), 2,4,6-trimethyloctane (CAS 62016-37-9), 2,5,6-trimethyloctane (CAS 62016-14-2), decane (CAS 124-18-5), 2-methylnonane (CAS 871-83-0), 4-methylnonane (CAS 17301-94-9), 3-methylnonane (CAS 5911-04-6), 3-ethyloctane (CAS 5881 -17-4), 2,2-dimethyloctane (CAS 15869-87-1), 2,3-dimethyloctane (CAS 7146-60-3), 2,5-dimethyloctane (CAS 15869-89- 3), 3,5-dimethyloctane (CAS 15869-93-9), 4-propyl heptane (CAS 3178-29-8), 3-ethyl-2-methylheptane (CAS 14676-29-0), 2,2,3-trimethylheptane (CAS 52896-92-1), 2,3,5 trimethylheptane (CAS 20278-85-7), 2,3,6-trimethylheptane (CAS 4032-93- 3), 3,3,4-trimethylheptane (CAS 20278-87-9), 2,3,4 trimethylheptane (CAS 52896-95-4), 2,2,4 trimethylheptane (CAS: 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), nonane (111-84-2), 2-methyloctane (3221-61-2), 3-methyloctane (CAS 2216-33-3), 4-methyloctane (CAS 2216-34-4), 2,4-dimethylheptane (CAS 2213-23-2), 2,6-dimethylheptane (CAS 1072-05-5), 2,3-dimethylheptane (CAS 3074 -71-3), 2,5-dimethylheptane (CAS 2216-30-0), 2,2-dimethylheptane (CAS 1071-26-7), 2,2,5-trimethylhexane (CAS 3522-94-9), 2,3,5-trimethylhexane (CAS 1069-53-0), 2,2,4-trimethylhexane (CAS 167476-26-5), 2,3,4 trimethylhexane (CAS 921-47- 1), 3-ethylheptane (CAS 15869-80-4), 4-ethylheptane (CAS 2216-32-2), octane (CAS 111-65-9), 2,2,4-trimethylheptane (CAS 592 -27-8), 3-methylheptane (CAS 589-81-1), 2-methylheptane (CAS 592-27-8), 2,3,4-trimethylpentane (CAS 565-75-3), 2,4-dimethylhexane (CAS 589-43-5), 2,5-dimethylhexane (CAS 592-13-2), 3,4-dimethylhexane (CAS 583-48-2), 3-ethylhexane (CAS 619-99 -8), 4-methylheptane (CAS 589-53-7), and mixtures thereof.

In one embodiment of the method according to the invention, the composition is any one of the compositions according to the invention and/or the aerosol according to the invention.

In one embodiment, the method comprises at least one step of applying a composition comprising:
a) at least one alkane of the formula CₙH₂ₙ₊₂, where n≤12, in a quantity of from 40% to 95% (v/v) by volume relative to the total volume of the composition and at least oxoester of formula II:

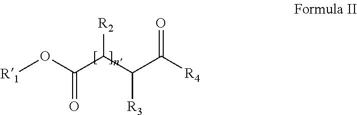

in which
R′₁ is selected from the group consisting of linear or branched alkyls comprising from 1 to 4 carbon atoms;
R₂, R₃, and R₄, which are identical or different, are selected from the group consisting of the hydrogen atom or linear or branched alkyls comprising from 1 to 4 carbon atoms; and
n′ is a natural number between 1 and 4,
   the content of said oxoester being from 5% to 60% (v/v) by volume relative to the total volume of the composition according to the invention.

In one embodiment, the method includes applying a composition comprising
a) at least one alkane of the formula CₙH₂ₙ₊₂, where n≤12, in a quantity of from 40% to 95% (v/v) by volume relative to the total volume of the composition and at least one oxoester of formula I,

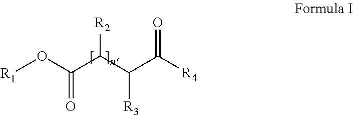

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
the content of said oxoester being from 5% to 60% (v/v) by volume relative to the total volume of the composition according to the invention.
b) at least one oil.

In one embodiment, the method comprises at least one step of applying a composition comprising:
a) at least one alkane of the formula CₙH₂ₙ₊₂, where 8≤n≤12, in a quantity of from 40% to 95% (v/v) by volume relative to the total volume of the composition and
b) at least oxoester of formula II:

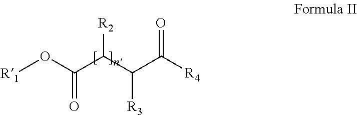

in which
R′₁ is selected from the group consisting of linear or branched alkyls comprising from 1 to 4 carbon atoms;
R₂, R₃, and R₄, which are identical or different, are selected from the group consisting of the hydrogen atom or linear or branched alkyls comprising from 1 to 4 carbon atoms; and
n′ is a natural number between 1 and 4,
   the content of said oxoester being from 5% to 60% (v/v) by volume relative to the total volume of the composition according to the invention.

In one embodiment, the method comprises at least one step of applying a composition comprising:
c) at least one alkane of the formula CₙH₂ₙ₊₂, where 8≤n≤12, in a quantity of from 40% to 95% (v/v) by volume relative to the total volume of the composition and
d) at least oxoester of formula II:

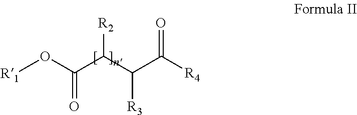

in which
R′₁ is selected from the group consisting of linear or branched alkyls comprising from 1 to 4 carbon atoms;
R₂, R₃, and R₄, which are identical or different, are selected from the group consisting of the hydrogen atom or linear or branched alkyls comprising from 1 to 4 carbon atoms; and
n′ is a natural number between 1 and 4,
   the content of said oxoester being from 5% to 60% (v/v) by volume relative to the total volume of the composition according to the invention,
   and in which the alkane of the formula CₙH₂ₙ₊₂, where 8≤n≤12, is linear or branched and selected from the group consisting of dodecane, undecane, decane, nonane, and/or an octane.

In one embodiment, the method comprises at least one step of applying a composition comprising:
a) at least one alkane of the formula CₙH₂ₙ₊₂, where 8≤n≤12, in a quantity of from 40% to 95% (v/v) by volume relative to the total volume of the composition and
b) at least oxoester of formula II:

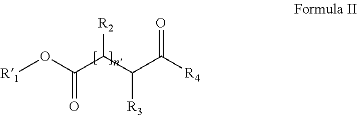

in which
R′₁ is selected from the group consisting of linear or branched alkyls comprising from 1 to 4 carbon atoms;
R₂, R₃, and R₄, which are identical or different, are selected from the group consisting of the hydrogen atom or linear or branched alkyls comprising from 1 to 4 carbon atoms; and
n′ is a natural number between 1 and 4,
   the content of said oxoester being from 5% to 60% (v/v) by volume relative to the total volume of the composition according to the invention,
   and in which the alkane of the formula CₙH₂ₙ₊₂, where 8≤n≤12, is selected from the group comprising: dodecane, 2-methylundecane (CAS 7045-71-8), 3-methylundecane (CAS 1002 -43-3), 4-methylundecane (CAS 2980-69-0), 5-methylundecane (CAS 1632-70-8), 6-methylundecane (CAS 17302-33-9), 2,4-dimethyldecane (CAS 2801-84-5) 4,4-dimethyldecane (CAS 17312-39-9), 3,5-dimethyldecane (17312-48-0), 2,5-dimethyldecane (CAS 17312-50- 4), 2,3-dimethyldecane (17312-44-6), 3,3-dimethyldecane (17302-38-4), 3,7-dimethyldecane (CAS 17312-54-8), 3,4,6-trimethylnonane (CAS 62184-24-1) 3,5,6-trimethylnonane (CAS 62184-26-3), 3,5,7-trimethylnonane (CAS 62184-27-4), 2.5, 7-trimethylnonane (CAS 62184-14-9), 2,5,6-trimethylnonane (CAS 62184-13-8), 2,5,7-trimethylnonane (CAS 62184-14-9), 2.5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7), 3-ethyl-4-methylnonane (CAS 62184-45-6), 3-ethyl-4,5-dimethyloctane (CAS 62183-72-6), 2,5-dimethyl-6-ethyloctane (CAS 62183-50-0), undecane (CAS 1120-21-4), 2-methyldecane (CAS 6975 -98-0), 4-methyldecane (CAS 2847-72-5), 3-methyldecane (13141-34-3), 5-methyldecane (CAS 13151-35-4), 2-6-dimethylnonane (CAS 17302-28-2), 3,7-dimethylnonane (CAS 17302-32-8), 4,5-dimethylnonane (CAS 17302-23-7), 2,3-dimethylnonane (CAS 2884- 06-2), 2,4,6-trimethyloctane (CAS 62016-37-9), 2,5,6-trimethyloctane (CAS 62016-14-2), decane (CAS 124-18-5), 2-methylnonane (CAS 871-83-0), 4-methylnonane (CAS 17301-94-9), 3-methylnonane (CAS 5911-04-6), 3-ethyloctane (CAS 5881 -17-4), 2,2-dimethyloctane (CAS 15869-87-1), 2,3-dimethyloctane (CAS 7146-60-3), 2,5-dimethyloctane (CAS 15869-89-3), 3,5-dimethyloctane (CAS 15869-93-9), 4-propyl heptane (CAS 3178-29-8), 3-ethyl-2-methylheptane (CAS 14676-29-0), 2,2,3-trimethylheptane (CAS 52896-92-1), 2,3,5 trimethylheptane (CAS 20278-85-7), 2,3,6-trimethylheptane (CAS 4032-93- 3), 3,3,4-trimethylheptane (CAS 20278-87-9), 2,3,4 trimethylheptane (CAS 52896-95-4), 2,2,4 trimethylheptane (CAS: 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), nonane (111-84-2), 2-methyloctane (3221-61-2), 3-methyloctane (CAS 2216-33-3), 4-methyloctane (CAS 2216-34-4), 2,4-dimethylheptane (CAS 2213-23-2), 2,6-dimethylheptane (CAS 1072-05-5), 2,3-dimethylheptane (CAS 3074 -71-3), 2,5-dimethylheptane (CAS 2216-30-0), 2,2-dimethylheptane (CAS 1071-26-7), 2,2,5-trimethylhexane (CAS 3522-94- 9), 2,3,5-trimethylhexane (CAS 1069-53-0), 2,2,4-trimethylhexane (CAS 167476-26-5), 2,3,4 trimethylhexane (CAS 921-47- 1), 3-ethylheptane (CAS 15869-80-4), 4-ethylheptane (CAS 2216-32-2), octane (CAS 111-65-9), 2,2,4-trimethylheptane (CAS 592 -27-8), 3-methylheptane (CAS 589-81-1), 2-methylheptane (CAS 592-27-8), 2,3,4-trimethylpentane (CAS 565-75-3), 2,4-dimethylhexane (CAS 589-43-5), 2,5-dimethylhexane (CAS 592-13-2), 3,4-dimethylhexane (CAS 583-48-2), 3-ethylhexane (CAS 619-99 -8), 4-methylheptane (CAS 589-53-7), and mixtures thereof.

In one embodiment, the method includes applying a composition comprising
a) at least one alkane of the formula CₙH₂ₙ₊₂, where 8≤n≤12, in a quantity of from 40% to 95% (v/v) by volume relative to the total volume of the composition and
b) at least one oxoester of formula I,

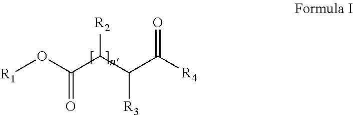

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
the content of said oxoester being from 5% to 60% (v/v) by volume relative to the total volume of the composition according to the invention.
c) at least one oil.

In one embodiment, the method includes applying a composition comprising
a) at least one alkane of the formula CₙH₂ₙ₊₂, where 8≤n≤12, in a quantity of from 40% to 95% (v/v) by volume relative to the total volume of the composition and
b) at least one oxoester of formula I,

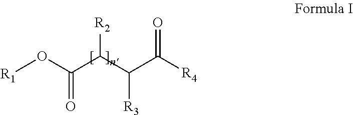

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
the content of said oxoester being from 5% to 60% (v/v) by volume relative to the total volume of the composition according to the invention.
c) at least one oil,
   and in which the alkane of the formula CₙH₂ₙ₊₂, where 8≤n≤12, is linear or branched and selected from the group consisting of dodecane, undecane, decane, nonane, and/or an octane.

In one embodiment, the method includes applying a composition comprising
a) at least one alkane of the formula CₙH₂ₙ₊₂, where 8≤n≤12, in a quantity of from 40% to 95% (v/v) by volume relative to the total volume of the composition and
b) at least one oxoester of formula I,

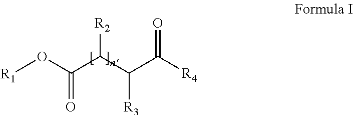

in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
the content of said oxoester being from 5% to 60% (v/v) by volume relative to the total volume of the composition according to the invention.
c) at least one oil,
   and in which the alkane of the formula CₙH₂ₙ₊₂, where 8≤n≤12, is selected from the group comprising: dodecane, 2-methylundecane (CAS 7045-71-8), 3-methylundecane (CAS 1002 -43-3), 4-methylundecane (CAS 2980-69-0), 5-methylundecane (CAS 1632-70-8), 6-methylundecane (CAS 17302-33-9), 2,4-dimethyldecane (CAS 2801-84-5) 4,4-dimethyldecane (CAS 17312-39-9), 3,5-dimethyldecane (17312-48-0), 2,5-dimethyldecane (CAS 17312-50- 4), 2,3-dimethyldecane (17312-44-6), 3,3-dimethyldecane (17302-38-4), 3,7-dimethyldecane (CAS 17312-54-8), 3,4,6-trimethylnonane (CAS 62184-24-1) 3,5,6-trimethylnonane (CAS 62184-26-3), 3,5,7-trimethylnonane (CAS 62184-27-4), 2.5, 7-trimethylnonane (CAS 62184-14-9), 2,5,6-trimethylnonane (CAS 62184-13-8), 2,5,7-trimethylnonane (CAS 62184-14-9), 2.5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetram ethyloctane (CAS 35866-96-7), 3-ethyl-4-methylnonane (CAS 62184-45-6), 3-ethyl-4,5-dimethyloctane (CAS 62183-72-6), 2,5-dimethyl-6-ethyloctane (CAS 62183-50-0), undecane (CAS 1120-21-4), 2-methyldecane (CAS 6975 -98-0), 4-methyldecane (CAS 2847-72-5), 3-methyldecane (13141-34-3), 5-methyldecane (CAS 13151-35-4), 2-6-dimethylnonane (CAS 17302-28-2), 3,7-dimethylnonane (CAS 17302-32-8), 4,5-dimethylnonane (CAS 17302-23-7), 2,3-dimethylnonane (CAS 2884- 06-2), 2,4,6-trimethyloctane (CAS 62016-37-9), 2,5,6-trimethyloctane (CAS 62016-14-2), decane (CAS 124-18-5), 2-methylnonane (CAS 871-83-0), 4-methylnonane (CAS 17301-94-9), 3-methylnonane (CAS 5911-04-6), 3-ethyloctane (CAS 5881 -17-4), 2,2-dimethyloctane (CAS 15869-87-1), 2,3-dimethyloctane (CAS 7146-60-3), 2,5-dimethyloctane (CAS 15869-89- 3), 3,5-dimethyloctane (CAS 15869-93-9), 4-propyl heptane (CAS 3178-29-8), 3-ethyl-2-methylheptane (CAS 14676-29-0), 2,2,3-trimethylheptane (CAS 52896-92-1), 2,3,5 trimethylheptane (CAS 20278-85-7), 2,3,6-trimethylheptane (CAS 4032-93- 3), 3,3,4-trimethylheptane (CAS 20278-87-9), 2,3,4 trimethylheptane (CAS 52896-95-4), 2,2,4 trimethylheptane (CAS: 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), nonane (111-84-2), 2-methyloctane (3221-61-2), 3-methyloctane (CAS 2216-33-3), 4-methyloctane (CAS 2216-34-4), 2,4-dimethylheptane (CAS 2213-23-2), 2,6-dimethylheptane (CAS 1072-05-5), 2,3-dimethylheptane (CAS 3074 -71-3), 2,5-dimethylheptane (CAS 2216-30-0), 2,2-dimethylheptane (CAS 1071-26-7), 2,2,5-trimethylhexane (CAS 3522-94- 9), 2,3,5-trimethylhexane (CAS 1069-53-0), 2,2,4-trimethylhexane (CAS 167476-26-5), 2,3,4 trimethylhexane (CAS 921-47- 1), 3-ethylheptane (CAS 15869-80-4), 4-ethylheptane (CAS 2216-32-2), octane (CAS 111-65-9), 2,2,4-trimethylheptane (CAS 592 -27-8), 3-methylheptane (CAS 589-81-1), 2-methylheptane (CAS 592-27-8), 2,3,4-trimethylpentane (CAS 565-75-3), 2,4-dimethylhexane (CAS 589-43-5), 2,5-dimethylhexane (CAS 592-13-2), 3,4-dimethylhexane (CAS 583-48-2), 3-ethylhexane (CAS 619-99 -8), 4-methylheptane (CAS 589-53-7), and mixtures thereof

In one embodiment of any one of the methods according to the invention, the at least one alkane of the formula CₙH₂ₙ₊₂, where 8≤n≤12, is a bioalkane.

In one embodiment of any one of the methods according to the invention, the alkanes of the formula CₙH₂ₙ₊₂, where 8≤n≤12, comprise a mixture of decane and dodecane.

In one embodiment of any one of the methods according to the invention, the at least one oxoester is a levulinate.

In one embodiment of any one of the methods according to the invention, the at least one oxoester is a butyl levulinate and/or ethyl levulinate.

In one embodiment of any one of the methods according to the invention, the composition also comprises at least one oil of vegetable origin.

In one embodiment of any one of the methods according to the invention, the oil content is less than or equal to 30% (v/v) by volume of oil relative to the total volume of the composition according to the invention.

In one embodiment, the surface of an object is a surface of a metallic object.

In one embodiment, the object comprises all or part of an electronic component, a mechanical part, an automobile part, a cable, a chain, and/or a tool.

In one embodiment, the method makes it possible to protect the surface against humidity, rust, oxidation, and/or freezing.

In one embodiment, application is performed by dipping, spraying, and/or aerosol spraying.

In one embodiment, the application has a duration of less than 60 minutes.

In one embodiment, the application has a duration of less than 15 minutes.

### DESCRIPTION OF THE FIGURES

FIGS. 1 to 5 are photographs illustrating the evolution over time of the degreasing effect on burnt grease through soaking in formulas 1 to 4 or WD40, respectively in that order, according to Example 2.

FIGS. 6 to 10 are photographs illustrating the evolution over time of the degreasing effect on burnt grease through spraying of formulas 1 to 4 or WD40, respectively in that order, according to Example 3.

FIGS. 11 to 16 are photographs illustrating the evolution over time of the degreasing effect on baked grease through spraying of formulas 1 to 4 or WD40, respectively in that order, according to Example 4.

FIG. 17 is a curve showing the evolution of the percentage of evaporation as a function of time according to Example 6, with—from the curve having the fastest growth to the slowest—formula 6, WD40®, formula 5, formula 4, formula 3, and formula 1.

In the examples, all percentages are by volume unless otherwise indicated, temperature is in degrees Celsius unless otherwise indicated, and pressure is atmospheric pressure unless otherwise indicated.

### EXAMPLES

### Example 1: Preparation of Compositions According to the Invention

The compositions were prepared by mixing in a beaker at room temperature in the proportions as described in Table 1.

Vegelight® Silk, marketed by BIOSYNTHIS, is a mixture of alkanes of vegetable origin of the formula CₙH₂ₙ₊₂, where n≤12, comprising 17% (v/v) by volume of decane and 83% (v/v) by volume of dodecane relative to the total weight of the alkane mixture.

TABLE 1

| | Ethyl levulinate | Butyl levulinate | Vegelight ® | Iso C10 | Dodecene | Coconut oil |
|---|---|---|---|---|---|---|
| | (in % v/v) | (in % v/v) | Silk | (in % v/v) | (in % v/v) | (in % v/v) |
| Formula 1 | 50 | 50 | | | | |
| (Comparative)r1 | | | | | | |
| Formula 2 | 5 | 5 | 90 | | | |
| Formula 3 | 25 | 25 | 50 | | | |
| Formula 4 | 5 | 5 | 75 | | | 15 |
| Formula 5 | 15 | 15 | | | 55 | 15 |
| (Comparative) | | | | | | |
| Formula 6 | 15 | 15 | | 55 | | 15 |

### Example 2: Evaluation of the Degreasing Effect through Soaking of Burnt Grease in the Composition According to the Invention

Stainless steel plates were soaked in lithium grease.

The deposited lithium grease was then burned using a blowtorch flame.

The plates were then placed in test tubes containing one of the formulas according to Example 1 or WD40®.

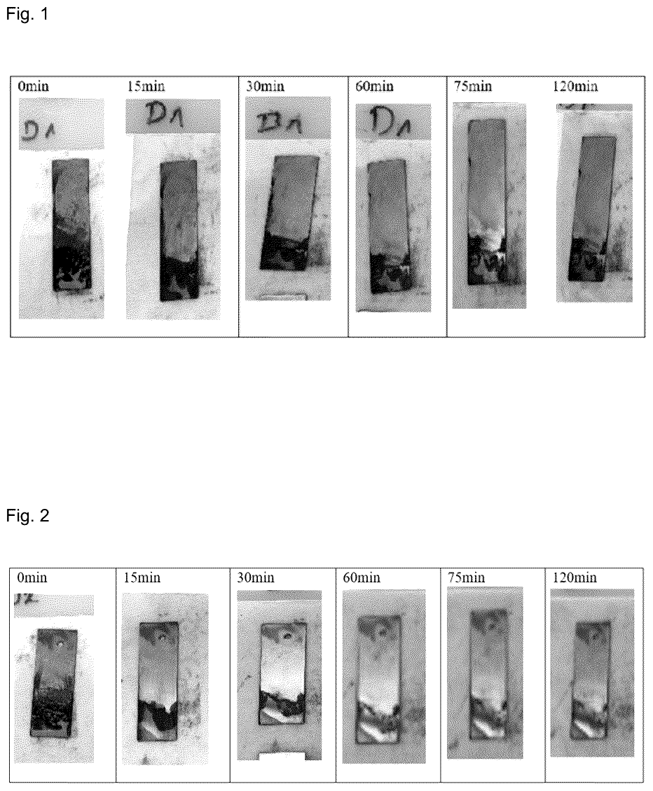

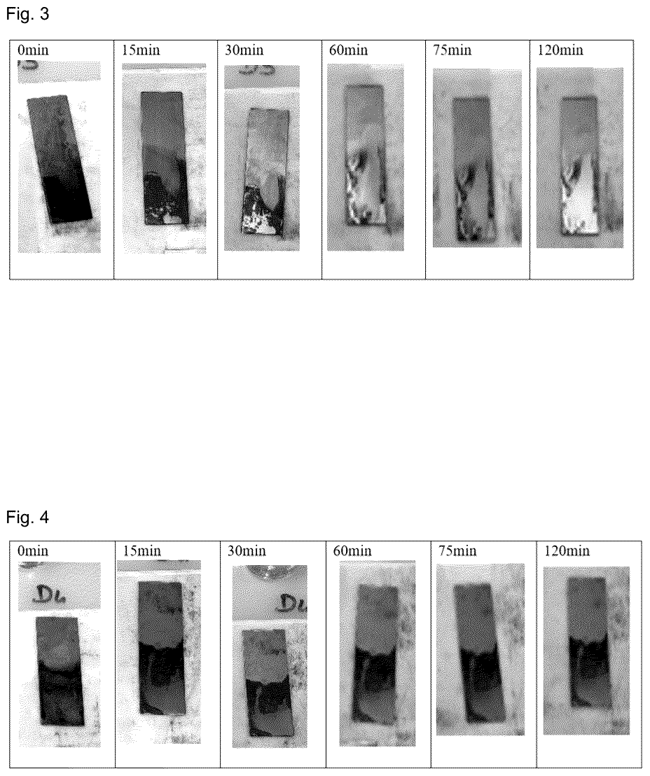

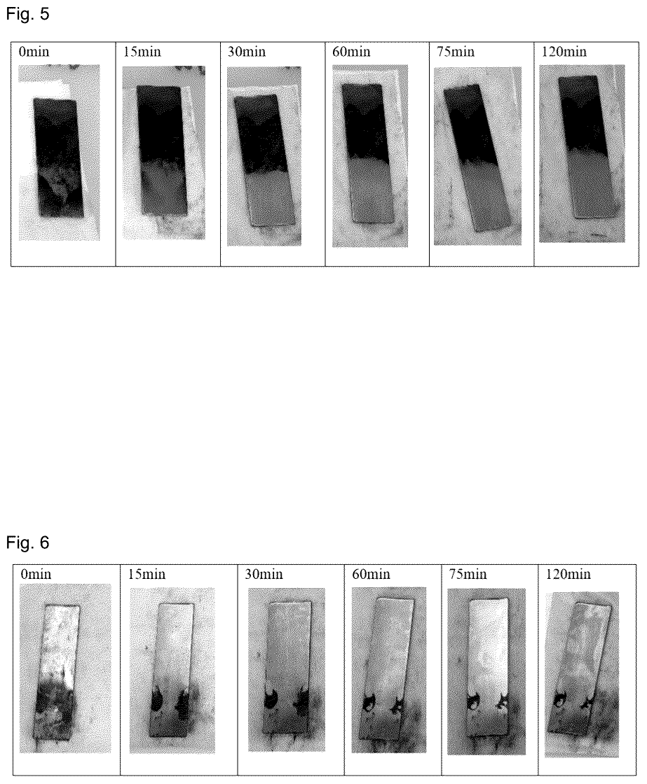

The plates were finally photographed at t=0 min, 15 min, 30 min, 60 min, 75 min, 120 min as presented in FIGS. 1 to 5 in the respective order of appearance in the table below.

The times after which the degreasing effect was observed for the first time are listed in Table 2.

TABLE 2

| | Time (in mins) |
|---|---|
| Formula 1 (comparative) | No degreasing effect |
| Formula 2 | 60 |
| Formula 3 | 30 |
| Formula 4 | 15 |
| WD40 ® | 15 |

Conclusion: Formula 1, corresponding to the use of oxoester alone, did not enable a degreasing effect to be observed through soaking. With formula 2, when the proportion of alkane of the formula CₙH₂ₙ₊₂, where n≤12, is at least equivalent to the proportion of oxoester, a degreasing effect is observed after 60 minutes. With formula 3, this effect is observed more and more rapidly as the amount of alkane increases. With formula 4, the presence of a vegetable oil enables a degreasing effect to be achieved within a period comparable to that achieved with the formulation of reference, WD40®.

### Example 3: Evaluation of the Degreasing Effect on Burnt Grease through Spraying of the Composition According to the Invention

Stainless steel plates were soaked in lithium grease.

The deposited lithium grease was then burned using a blowtorch flame.

The formulas according to Example 1 or WD40® were then applied to the plates by spraying in equivalent quantities.

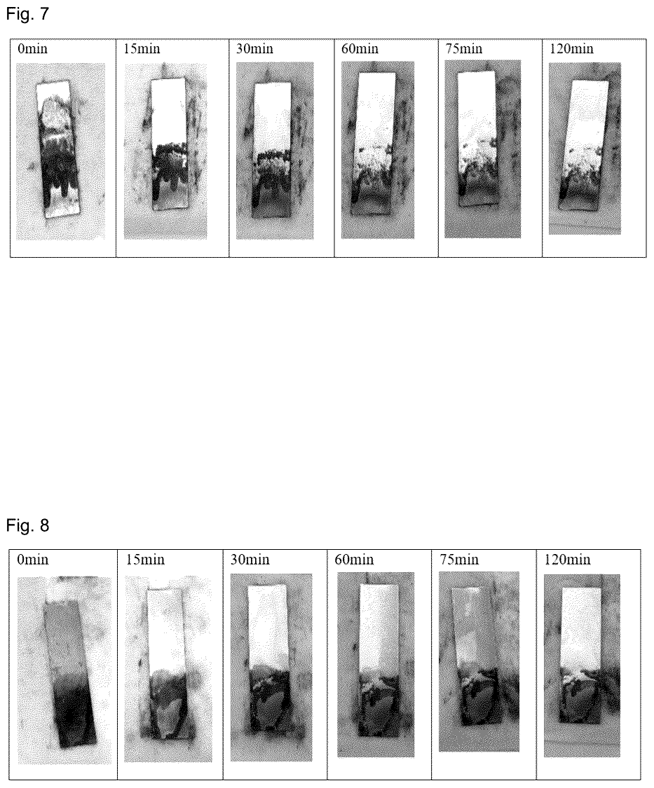

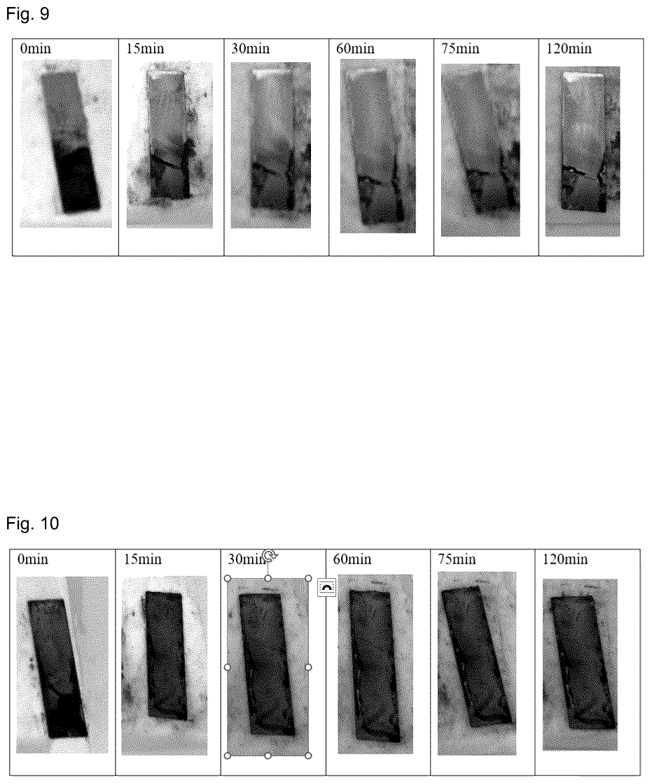

The plates were finally photographed at t=0 min, 15 min, 30 min, 60 min, 75 min, 120 min as presented in FIGS. 6 to 10 in the respective order of appearance in the table below.

The times after which the degreasing effect was observed for the first time are listed in Table 3.

TABLE 3

| | Time (in mins) |
|---|---|
| Formula 1 (Comparative) | 15 |
| Formula 2 | 120 |
| Formula 3 | 120 |
| Formula 4 | 15 |
| WD40 ® | 15 |

Conclusion: Formula 1 and formula 4 enable a degreasing effect to be achieved within a period comparable to that achieved with the formulation of reference, WD40®.
Formulas 2 and 3 enable a degreasing effect to be achieved, but over a much longer period.

### Example 4: Evaluation of the Degreasing Effect on Baked Grease through Spraying of the Composition According to the Invention

Stainless steel plates were soaked in lithium grease.

The deposited lithium grease was then baked at 300° C.

The formulas according to Example 1 or WD40® were then applied to the plates by spraying in equivalent quantities.

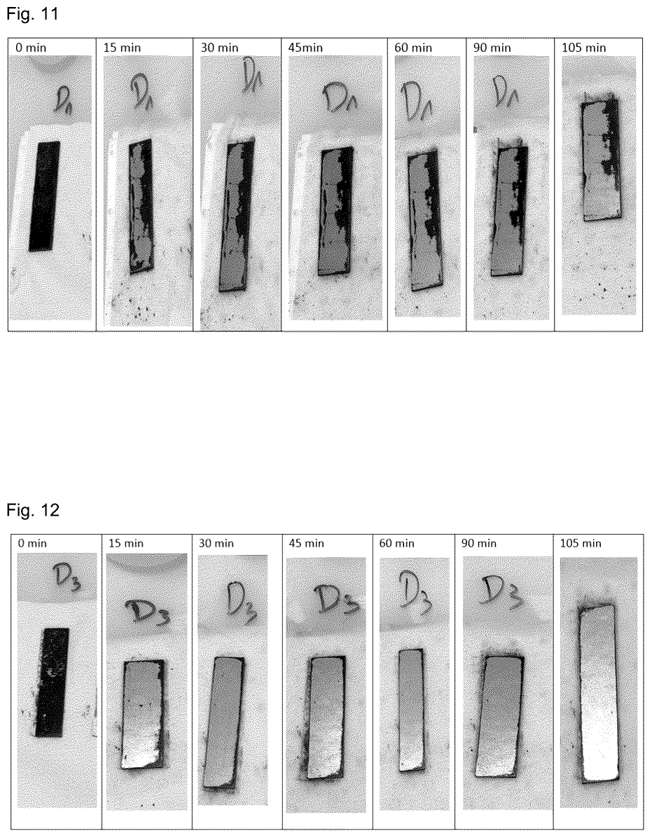

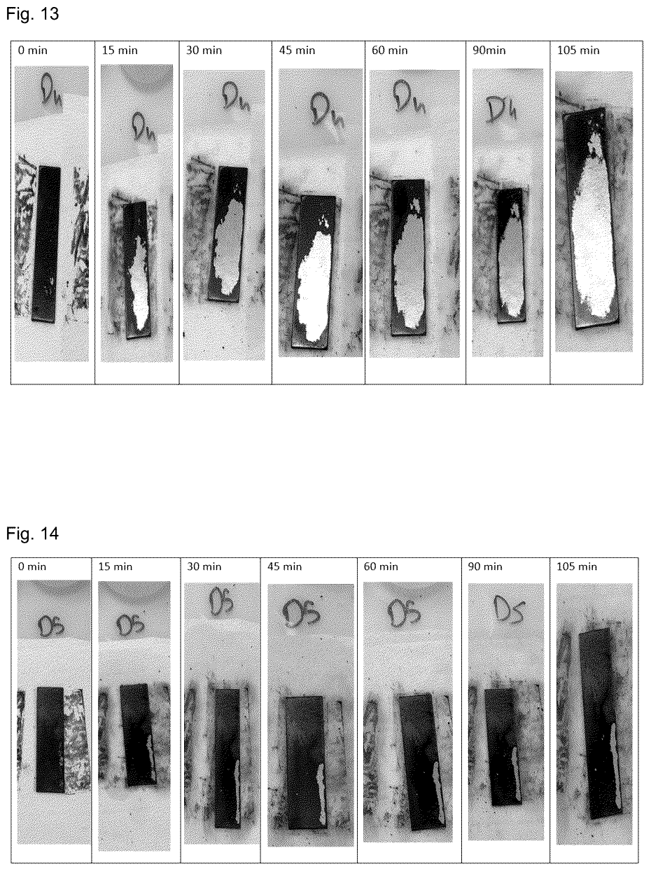

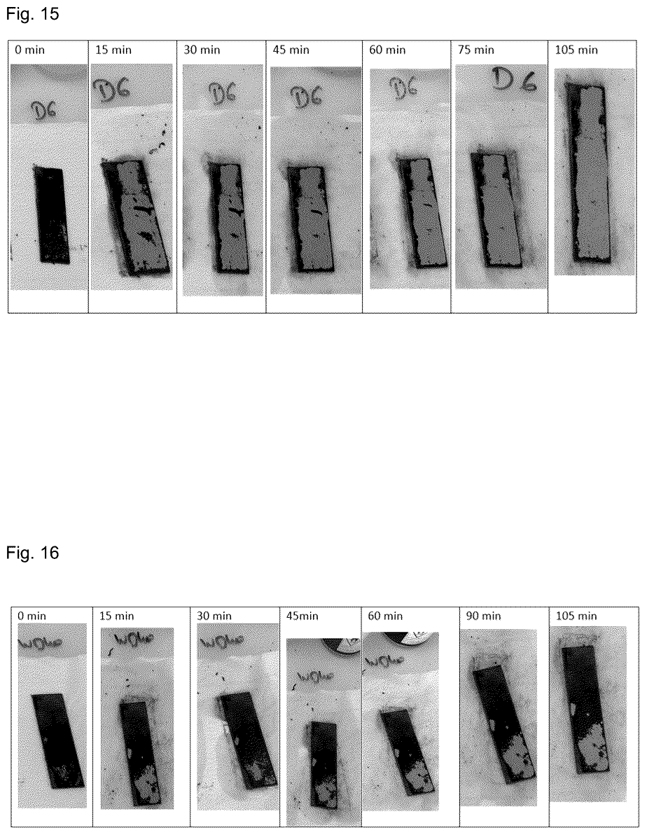

The plates were finally photographed at t=0 min, 15 min, 30 min, 60 min, 75 min, 120 min as presented in FIGS. 11 to 16 in the respective order of appearance in the table below.

The times after which the degreasing effect was observed for the first time are given in the table and in the figures.

TABLE 4

| | Time (in mins) |
|---|---|
| Formula 1 (Comparative) | 15 |
| Formula 3 | 15 |
| Formula 4 | 15 |
| Formula 5 (Comparative) | No degreasing effect |
| Formula 6 | 15 |
| WD40 ® | No degreasing effect |

Conclusion: Formulas 3, 4, and 6 according to the invention demonstrated an effect comparable to that of formula 1, which comprises only oxoesters.

Conversely, formula 5, which comprises an alkene instead of an alkane, as well as WD40® showed no degreasing effect.

### Example 5: Measurements of the Viscosity and the Density of the Compositions According to the Invention

The density and the viscosities of formulas 1 to 6 and of WD40 were measured using a Brookfield viscometer (ULA module, speed 100 rpm).

The results are summarized in Table 5.

TABLE 5

| | Density | Viscosity | Viscosity |
|---|---|---|---|
| | (20° C. g/cm³) | (centipoises) | (centistokes) |
| Formula 1 | 0.9926 | 2.41 | 2.42 |
| (Comparative) | | | |
| Formula 2 | 0.7679 | 1.39 | 1.81 |
| Formula 3 | 0.7830 | 1.45 | 1.85 |
| Formula 4 | 0.7879 | 1.99 | 2.52 |
| Formula 5 | 0.8401 | 2.58 | 2.58 |
| (Comparative) | | | |
| Formula 6 | 0.8231 | 1.67 | 2.02 |
| WD40 ® | 0.8026 | 2.99 | 3.76 |

### Example 6: Evaluation of the Volatility of the Compositions According to the Invention

The percentage of evaporation as a function of time for the formulas according to Example 1 and WD40® was measured using a LABOMODERNE THERMOBALANCE KERN DBS 60-3 desiccator at 20° C. under a controlled atmosphere.

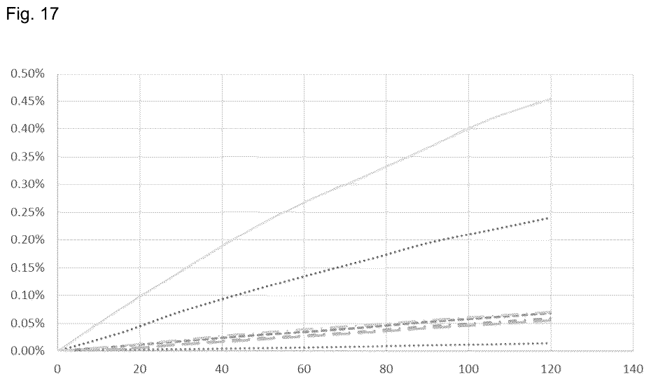

The evaporation curve obtained is presented in FIG. 17.

Formula 6 according to the invention is the most volatile. WD40® has the second-highest volatility. The formula comprising exclusively levulinate esters is the least volatile.

The compositions according to the invention therefore have greater volatility than the levulinate esters alone.

### Example 7: Evaluation of the Skin Compatibility of a Composition According to the Invention

A clinical test was carried out on a panel of 11 people aged 23 to 66 with the aim of verifying the skin compatibility of the product by applying an occlusive patch (Finn Chamber® AQUA Patch Test) containing a quantity of 0.02 ml of formula 4 according to Example 1 for 48 hours to the skin of the subject's back.

Thirty minutes after removal of the patch, a total absence of cutaneous manifestations (erythema, edema, dryness, desquamation, papules, vesicles, detergent effect, or reflectivity) was observed by the operator.

The composition according to the invention is therefore compatible with the skin.

1. A composition, wherein it comprises:
a) at least one alkane of the formula CₙH₂ₙ₊₂, where 8≤n≤12, in a quantity of from 40% to 95% (v/v) by volume relative to the total volume of the composition and
b) at least one oxoester of formula I, 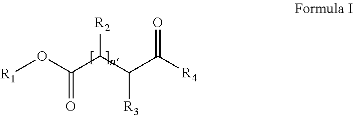
in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
the content of the oxoester being from 5% to 60% (v/v) by volume relative to the total volume of the composition.

2. The composition as set forth in claim 1, wherein at least one alkane of the formula CₙH₂ₙ₊₂, where 8≤n≤12, is linear or branched and selected from the group consisting of dodecane, undecane, decane, nonane, and/or an octane.

3. The composition as set forth in claim 1, wherein at least one alkane of the formula CₙH₂ₙ₊₂, where 8≤n≤12, is selected from the group comprising: dodecane, 2-methylundecane (CAS 7045-71-8), 3-methylundecane (CAS 1002-43-3), 4-methylundecane (CAS 2980-69-0), 5-methylundecane (CAS 1632-70-8), 6-methylundecane (CAS 17302-33 -9), 2,4-dimethyldecane (CAS 2801-84-5) 4,4-dimethyldecane (CAS 17312-39-9), 3,5-dimethyldecane (17312-48-0), 2.5-dimethyldecane (CAS 17312-50-4), 2,3-dimethyldecane (17312-44-6), 3,3-dimethyldecane (17302-38-4), 3,7-dimethyldecane (CAS 17312- 54-8), 3,4,6-trimethylnonane (CAS 62184-24-1) 3,5,6-trimethylnonane (CAS 62184-26-3), 3,5,7-trimethylnonane (CAS 62184- 27-4), 2,5,7-trimethylnonane (CAS 62184-14-9), 2,5,6-trimethylnonane (CAS 62184-13-8), 2,5,7-trimethylnonane (CAS 62184 -14-9), 2,5,8-trimethylnonane (CAS 49557-09-7), 3,3,4,5-tetramethyloctane (CAS 62185-21-1), 2,3,4,5- tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7), 3-ethyl-4-methylnonane (CAS 62184-45 -6), 3-ethyl-4,5-dimethyloctane (CAS 62183-72-6), 2,5-dimethyl-6-ethyloctane (CAS 62183-50-0), undecane (CAS 1120-21-4), 2-methyldecane (CAS 6975-98-0), 4-methyldecane (CAS 2847-72-5), 3-methyldecane (13141-34-3), 5-methyldecane (CAS 13151 -35-4), 2-6-dimethylnonane (CAS 17302-28-2), 3,7-dimethylnonane (CAS 17302-32-8), 4,5-dimethylnonane (CAS 17302-23-7), 2,3-dimethylnonane (CAS 2884-06-2), 2,4,6-trimethyloctane (CAS 62016-37-9), 2,5,6-trimethyloctane (CAS 62016-14-2), decane (CAS 124-18-5), 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3-dimethyloctane (CAS 7146-60-3), 2,5 -dimethyloctane (CAS 15869-89-3), 3,5-dimethyloctane (CAS 15869-93-9), 4-propyl heptane (CAS 3178-29-8), 3-ethyl-2-methylheptane (CAS 14676-29-0), 2,2,3-trimethylheptane (CAS 52896-92-1), 2,3,5 trimethylheptane (CAS 20278-85-7), 2,3,6 -trimethylheptane (CAS 4032-93-3), 3,3,4-trimethylheptane (CAS 20278-87-9), 2,3,4 trimethylheptane (CAS 52896-95-4), 2,2,4 trimethylheptane (CAS: 14720-74-2) 3,3-diethylhexane (CAS 17302-02-2), 2,2,3,3-tetramethylhexane (CAS 13475-81-5), 3-ethyl-2,2,3-trimethylpentane (CAS 52897-17-3), nonane (111-84-2), 2-methyloctane (3221-61-2), 3-methyloctane (CAS 2216-33-3), 4-methyloctane (CAS 2216-34-4), 2,4-dimethylheptane (CAS 2213-23-2), 2,6-dimethylheptane (CAS 1072-05-5), 2,3-dimethylheptane (CAS 3074-71-3), 2,5-dimethylheptane (CAS 2216-30-0), 2,2-dimethylheptane (CAS 1071-26-7), 2,2,5-trimethylhexane (CAS 3522-94- 9), 2,3,5-trimethylhexane (CAS 1069-53-0), 2,2,4-trimethylhexane (CAS 167476-26-5), 2,3,4 trimethylhexane (CAS 921-47- 1), 3-ethylheptane (CAS 15869-80-4), 4-ethylheptane (CAS 2216-32-2), octane (CAS 111-65-9), 2,2,4-trimethylheptane (CAS 592 -27-8), 3-methylheptane (CAS 589-81-1), 2-methylheptane (CAS 592-27-8), 2,3,4-trimethylpentane (CAS 565-75-3), 2,4-dimethylhexane (CAS 589-43-5), 2,5-dimethylhexane (CAS 592-13-2), 3,4-dimethylhexane (CAS 583-48-2), 3-ethylhexane (CAS 619-99 -8), 4-methylheptane (CAS 589-53-7), and mixtures thereof.

4. The composition as set forth in claim 1, wherein at least one alkane of the formula CₙH₂ₙ₊₂, where 8≤n≤12, is a bioalkane.

5. The composition as set forth in claim 1, wherein the alkanes of the formula CₙH₂ₙ₊₂, where 8≤n≤12, comprise a mixture of decane and dodecane.

6. The composition as set forth in claim 1, wherein at least one oxoester is a levulinate.

7. The composition as set forth in claim 6, wherein at least one oxoester is a butyl levulinate and/or ethyl levulinate.

8. The composition as set forth in claim 1, wherein it further comprises at least one oil of vegetable origin.

9. The composition as set forth in claim 8, wherein the oil content is less than or equal to 30% (v/v) by volume of oil relative to the total volume of the composition.

10. An aerosol comprising a composition as set forth in claim 1 and a propellant gas.

11. A method for degreasing, cleaning, protecting, releasing, shining, and/or lubricating a surface of an object comprising at least one step of applying to the surface a composition comprising:
a) at least one alkane of the formula CₙH₂ₙ₊₂, where 8≤n≤12, and
b) at least one oxoester of formula I, 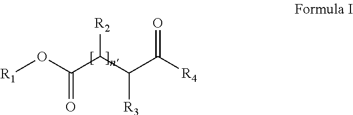
in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4.

12. The method as set forth in claim 11, wherein the composition is a composition comprising:
a) at least one alkane of the formula CₙH₂ₙ₊₂, where 8≤n≤12, in a quantity of from 40% to 95% (v/v) by volume relative to the total volume of the composition and
b) at least one oxoester of formula I, 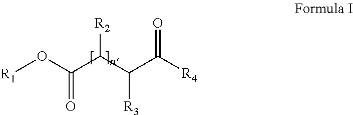
in which
R₁ is selected from the group consisting of linear or branched alkyls comprising from 1 to 8 carbon atoms;
R₂, R₃, and R₄, which are identical or different, are selected from the group consisting of the hydrogen atom and/or linear or branched alkyls comprising from 1 to 4 carbon atoms; and
n′ is a natural number from 1 to 4,
the content of the oxoester being from 5% to 60% (v/v) by volume relative to the total volume of the composition;
or an aerosol comprising the composition and a propellant gas.

13. The method as set forth in claim 11, wherein the surface is a surface of a metallic object.
